(19) United States
(12) Patent Application Publication
(10) Pub. No.: US 2016/0305919 A1
(43) Pub. Date: Oct. 20, 2016

(54) MICROFLUIDIC HPLC-CHIP FOR GLYCOPEPTIDE ANALYSIS WITH INTEGRATED HILIC ENRICHMENT

(71) Applicant: Agilent Technologies, Inc., Santa Clara, CA (US)

(72) Inventor: Gregory Staples, San Francisco, CA (US)

(21) Appl. No.: 15/053,491

(22) Filed: Feb. 25, 2016

Related U.S. Application Data
(62) Division of application No. 13/557,659, filed on Jul. 25, 2012, now abandoned.

(51) Int. Cl. G01N 30/60; G01N 1/40; B01D 15/32; C12Q 1/34; B01D 15/18; B01L 3/00; G01N 30/88

(52) U.S. Cl. CPC G01N 30/6095; B01L 3/502753; G01N 1/405; G01N 30/88; C12Q 1/34; C12Y 305/01052; B01D 15/1871; B01D 15/18; B01D 15/325; B01L 2200/0631; G01N 2030/8831; G01N 2030/8836; G01N 2333/98

(57) ABSTRACT

A microfluidic device for glycopeptide analysis includes an enrichment column capable of binding carbohydrates; a trapping column capable of binding peptides, wherein the trapping column is configured to be connected downstream of the enrichment column; a separation column, wherein the separation column is configured to be connected downstream of the trapping column; and a plurality of ports configured to work with a switching device to form a plurality of flow paths, wherein one of the plurality of flow paths allows the trapping column to be in fluid communication with the separation column. A method for glycopeptide analysis using a microfluidic device comprising a trapping column and a separation column, the method includes applying a sample of peptides to the microfluidic device; trapping the peptides on the trapping column; eluting the peptides from the trapping column into the separation column; and separating the peptides on the separation column.

### BACKGROUND OF INVENTION

1. Field of the Invention

The present invention relates to glycopeptide analysis, particularly microfluidic devices for glycopeptides analysis.

2. Background Art

Glycosylation is the most common post-translational modification of cell surface and extracellular matrix proteins. Glycoproteins play important roles in many cellular functions, such as cell-adhesion and immune responses. Changes in glycoprotein profiles have been correlated with altered physiological conditions and disease states, such as cancer and rheumatoid arthritis. Therefore, it is important to be able to characterize protein glycosylation states.

Glycoproteins may be glycosylated at different glycosylation sites (glycosites). Glycosylation typically occurs at asparagine (N-glycosylation) or serine (O-glycosylation) residues of the glycosites. At each potential glycosite, a glycan may or may not be attached to the protein. Even if glycans are present at a particular glycosite, different glycans (oligosaccharides) may be attached to the glycosite. This structural heterogeneity complicates the study of glycoproteins.

Structural studies of glycoproteins are often performed with mass spectrometry (MS). MS analysis of glycoproteins or glycopeptides has historically been a challenging task. One of the limitations to progress is the lack of robust, rapid, and simple tools for glycosite profiling. The difficulty in these analyses is mostly related to the unique properties of glycopeptides: 1) In a given protein digest, glycopeptides can be much lower in abundance than the non-glycopeptide counterparts; 2) Glycosylation sites are often occupied by a heterogeneous mixture of glycoforms, which lower their signal intensities in the MS mode (due to the distribution of the overall signal into several peaks); and 3) Electrospray ionization (which is commonly used for MS analysis of biomolecules) of glycopeptides is suppressed by non-glycopeptides that may be present in the same sample. In some cases, the presence of non-glycopeptides can completely suppress the ionization of glycopeptides, hence their MS signals. To deal with these difficulties, glycopeptides are typically purified from protein digests by a number of techniques, most often involving some type of tedious off-line solid-phase extraction. This approach is labor intensive and time consuming.

While glycopeptides may be analyzed as is (i.e., with the carbohydrates attached to the peptides), it is sometimes desirable to remove the carbohydrates on the glycopeptides so that it would be easier to characterize the carbohydrates and the peptide fragments separately. The process of removing carbohydrates from glycoproteins or glycopeptides (i.e., deglycosylation) traditionally involves an in-solution enzymatic reaction with PNGase F or a similar glycosidase that requires a long (e.g., 12 hours or longer) incubation time. The deglycosylation process is time-consuming and cumbersome, and the manual operations are error-prone.

### SUMMARY OF INVENTION

One aspect of the invention relates to microfluidic devices for glycopeptide analysis. A device in accordance with one embodiment of the invention includes a trapping column comprising a stationary phase capable of binding peptides; a separation column comprising a stationary phase capable of separating peptides; and a plurality of ports configured to work with a switching device to form a plurality of flow paths, wherein one of the plurality of flow paths allows the trapping column to be in fluid communication with the separation column. A device may further include an enrichment column capable of binding carbohydrates. The enrichment column may include a hydrophilic interaction (HILIC) stationary phase. The trapping column may include a hydrophilic interaction (HILIC) stationary phase, a reversed phase stationary phase, or a porous graphitic carbon (PGC) stationary phase. The separation column comprises a reversed-phase stationary phase or a porous graphitic carbon (PGC) stationary phase, wherein the reversed-phase stationary phase may be a C-18 silica-based stationary phase.

In accordance with some embodiments of the invention, a microfluidic device described above may further include a deglycosylation column comprising a solid support having a glycosidase immobilized thereto. The glycosidase may be one selected from PNGase F, β-N-Acetyl-glucosaminidase, α-Fucosidase, β-Galactosidase, α-Galactosidase, α-Neuraminidase, α-Mannosidase, β-Glucosidase, β-Xylosidase, β-Mannosidase, Endo F₁, Endo F₂, Endo F₃, or Endo H. Preferably, the glycosidase is PNGase F. In the above described device, the trapping column may be a polymer-based reversed phase column and the separation column is a silica-based reversed phase column.

In accordance with some embodiments of the invention, any of the above microfluidic devices may be part of a system for analyzing a sample, particularly a sample of glycoproteins or glycopeptides. The system may further include a switching device (e.g., a rotor or a rotary switch) and/or a mass spectrometer. A switching device (or a rotor) includes channels that can connect with inlet/outlet ports on the microfluidic device to form different flow paths.

Another aspect of the invention relates to methods for glycopeptide analysis using a microfluidic device comprising a trapping column and a separation column. A method in accordance with one embodiment of the invention includes applying a sample of peptides to the microfluidic device; trapping the peptides on the trapping column; eluting the peptides from the trapping column into the separation column; and separating the peptides on the separation column.

In accordance with some embodiments of the invention, the microfluidic device may further comprise an enrichment column, and the method further includes the step of enriching the peptides on the enrichment column prior to the trapping of the peptides on the trapping column. The enrichment column comprises a hydrophilic interaction (HILIC) stationary phase.

In accordance with some embodiments of the invention, the microfluidic device may further comprise a deglycosylation column comprising a solid support having a glycosidase immobilized thereto, and the method further comprising passing the sample of peptides through the deglycosylation column prior to the trapping of the peptides on the trapping columns. The glycosidase may be one selected from PNGase F, β-N-Acetyl-glucosaminidase, α-Fucosidase, β-Galactosidase, α-Galactosidase, α-Neuraminidase, α-Mannosidase, β-Glucosidase, β-Xylosidase, β-Mannosidase, Endo F₁, Endo F₂, Endo F₃, or Endo H. Preferably, the glycosidase is PNGase F. The trapping column is a polymer-based reversed phase column. The separation column is a silica-based reversed phase column.

Other aspects and advantages of the invention will be apparent from the following description and the appended claims.

### BRIEF DESCRIPTION OF DRAWINGS

FIG. 1 shows a flowchart of a method of glycoprotein analysis in accordance with one embodiment of the invention.

FIG. 2 shows a schematic of a microfluidic device for glycoprotein or glycopeptide analysis in accordance with one embodiment of the invention.

FIG. 3 shows a microfluidic device for glycoprotein or glycopeptide analysis and the operations of various states of the device in accordance with one embodiment of the invention.

FIG. 4 shows another microfluidic device for glycoprotein or glycopeptide analysis in accordance with one embodiment of the invention.

FIG. 5 shows a flowchart illustrating different methods of glycoprotein or glycopeptide analysis in accordance with one embodiment of the invention.

FIG. 6 shows another microfluidic device for glycoprotein or glycopeptide analysis that incorporates a deglycosylation reactor in accordance with one embodiment of the invention.

FIG. 7 shows results of glycopeptide analysis using a device of FIG. 3 in accordance with one embodiment of the invention.

FIG. 8 shows results of glycopeptide analysis using a device of FIG. 4 in accordance with one embodiment of the invention.

FIG. 9 shows the peptide sequence and the glycosylation sites of the peaks shown in FIG. 8.

FIG. 10 shows results of glycopeptide analysis using a device of FIG. 6 in accordance with one embodiment of the invention. (A) shows a total compound chromatogram of fetuin tryptic digest deglycosylated using a “heart-cut” approach. (B) shows a total compound chromatogram of fetuin tryptic digest with no deglycosylation. (C) shows mass spectra corresponding to the peptide containing site 1 in its deglycosylated form. (D) shows mass spectra corresponding to the peptide containing site 1 in its glycosylated form.

FIG. 11 shows results of glycopeptide analysis using a device of FIG. 6 in accordance with one embodiment of the invention.

FIG. 12 shows a reaction mechanism of a glycosidase reaction.

FIG. 13 shows mass spectrometry results of glycosidase reaction conducted in regular water, and mass spectrometry results of glycosidase reaction conducted in ¹⁸O enriched water in accordance with one embodiment of the invention.

### DEFINITION

A “microfluidic device” is a device comprising chambers and/or channels of micron or submicron dimensions that allow passage of fluid. The chambers or channels are generally 1 μm to less than 1000 μm in diameter (or, if not circular, the largest dimension of the cross section), such as 1 μm to 500 μm, 10 μm to 300 μm, 50 μm to 250 μm, by way of examples.

As used herein, the term “glycoprotein” refers to a naturally occurring protein having one or more carbohydrates attached thereto. As used herein, the term “glycopeptide” refers to a fragment of a glycoprotein having one or more carbohydrates attached thereto. “Glycopeptides” are typically generated from “glycoproteins” by protease cleavage. A “glycopeptide” may comprise a “peptide part” and one or more “carbohydrate parts.”

As used herein, the term “deglycosylated peptide” refers to a glycopeptide having been subjected to a deglycosylation reaction to remove its carbohydrates. In other words, a “deglycosylated peptide” originates from a glycopeptide, but has no or substantially no carbohydrate attached thereto. Deglycosylation reactions typically involve glycosidases, which are enzyme that removes carbohydrates from glycoproteins.

As used herein, the term “enrichment column” refers to a column on a microfluidic device having a stationary phase for binding carbohydrate parts on glycopeptides such that glycopeptides can be enriched in concentrations. Examples of “enrichment column” for use in embodiments of the invention include columns having a stationary phase for hydrophilic interaction chromatography (HILIC). Many HILIC stationary phases are known in the art and may be used with embodiments of the invention. The stationary phase of HILIC typically comprises polar materials, such as silica, cyano, amino, diol, etc.

As used herein, the term “trapping column” refers to a column on a microfluidic device having a stationary phase for binding either the carbohydrate parts or the peptide parts of glycopeptides such that peptides (e.g., glycopeptides and/or deglycosylated peptides) can be trapped or enriched in concentrations. When a “trapping column” is selected to bind the carbohydrate parts of glycopeptides, the trapping column may have a stationary phase for hydrophilic interaction chromatography (HILIC). However, the particular stationary phase used in the trapping column may or may not be the same as that used in the enrichment column described above. In accordance with some embodiments of the invention, the enrichment column and trapping column may be the same column—i.e., these two functions are accomplished by a single enrichment/trapping column. When a “trapping column” is selected to bind the peptide parts of glycopeptides, the trapping column may comprise a PGC or reversed phase stationary phase, wherein the reversed phase stationary phase may be polymer-based or silica-based. Any reversed phase stationary phase known in the art may be used, such as C-1, C-4, C-8, and C-18.

As used herein, the term “separation column” refers to an analytical column on a microfluidic device having a stationary phase for separating peptides (e.g., glycopeptides and/or deglycosylated peptides). The “separation column” may separate the peptides by electrophoresis or liquid chromatography (LC), particularly HPLC. Examples of HPLC “separation column” for use in embodiments of the invention include columns having a PGC or reversed phase stationary phase, wherein the reversed phase stationary phase may be polymer-based or silica-based. Any reversed phase stationary phase known in the art may be used, such as C-1, C-4, C-8, and C-18.

### DETAILED DESCRIPTION

Mapping of glycosylation sites in proteins is a difficult and time-consuming task that requires expert level knowledge. In a typical workflow, a glycoprotein or mixture of glycoproteins is digested with trypsin (or another protease) and then deglycosylated in solution using the enzyme PNGase F. As noted above, these manual processes are time-consuming and require a relatively large amount of samples.

Recently, Bynum et al. disclosed a microfluidic device for analyzing carbohydrates released from glycoproteins (U.S. Patent Application Publication No. 2010/0190146, the disclosure of which is incorporated by reference in its entirety). This microfluidic device is efficient and convenient for the analysis of carbohydrates released from glycoproteins. The device disclosed in the '146 application is similar to the Agilent mAb-Glyco Chip, which is designed for on-chip deglycosylation of monoclonal antibodies (mAbs) as well as subsequent on-chip enrichment, separation and MS based detection of cleaved N-glycans with an Agilent HPLC-Chip/MS system. Deglycosylation is based on an integrated immobilized PNGase F enzyme reactor column (Agilent Technologies, Santa Clara, Calif.).

Embodiments of the invention relate to microfluidic devices for glycopeptide analysis. Specifically, microfluidic devices of the invention are for the analysis of glycopeptide-derived peptides, with or without carbohydrates attached thereto. Such peptides may be glycopeptides generated from glycoproteins by protease cleavages. These glycopeptides may be analyzed “as is” (i.e., without removal of the carbohydrates). Alternatively, the glycopeptides may be further deglycosylated using glycosidases and then analyzed as deglycosylated peptides. These microfluidic devices provide convenient and efficient tools for the analysis of glycosites.

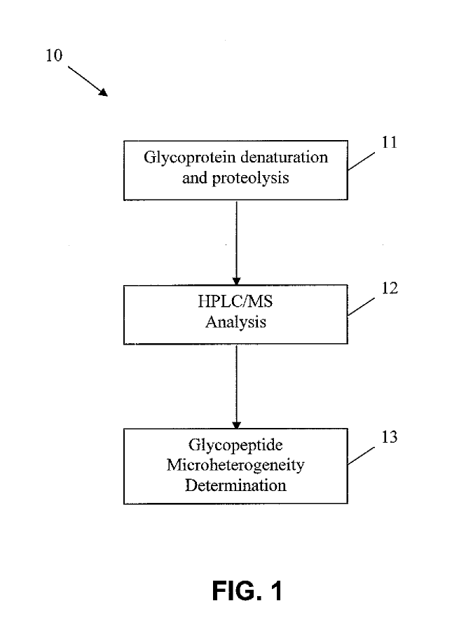

FIG. 1 shows a flowchart illustrating a general process for glycoprotein analysis. As shown, a method 10 may start with denaturing the glycoproteins and generating the peptide fragments (step 11). The denaturation of proteins may optionally involve reduction with a thiol reagent (e.g., DTT) or other reducing agents to break up the disulfide bonds. The reduced thiol (—SH) group may be alkylated (e.g., using iodoacetate or iodoacetamide) to prevent the reduced thiols from reforming the disulfide bonds. Note that denaturation of the glycoproteins may also be achieved with heat or other means. The denatured glycoproteins are then cleaved with proteases into smaller fragments to facilitate analysis.

The glycoprotein cleavages are typically performed with specific proteases, such as trypsin (specific hydrolysis of the peptide bonds at the carboxyl side of lysine or arginine) or Glu-C protease (specific for the peptide bonds on the carboxyl side of glutamic acid), that will produce predictable fragments. Other proteases that may also be used may include chymotrypsin, thrombin, enterokinase, Factor Xa, and the like.

The proteolytic fragments (peptides and glycopeptides) from the glycoproteins are then subjected to purification and identification, typically using chromatography and mass spectrometry (e.g., HPLC/MS) (step 12). Commonly used MS may include time-of-flight (TOF) MS or quadrupole-TOF (Q-TOF) MS. The MS analysis may also employ tandem MS/MS to obtain sequence information. Information obtained from MS analysis may be used to determine the glycosylation sites and the carbohydrate identities (step 13).

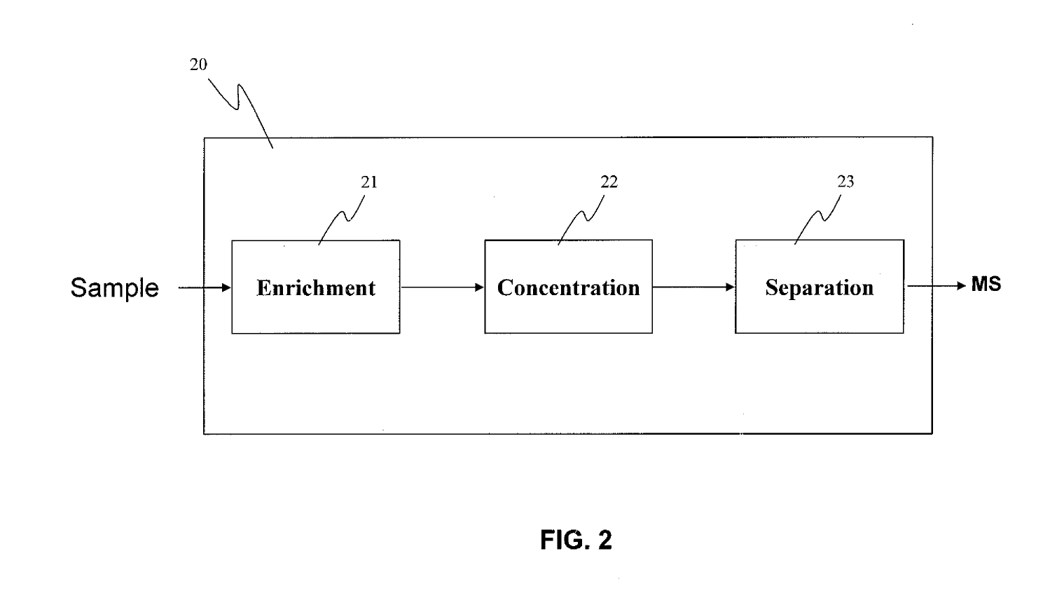

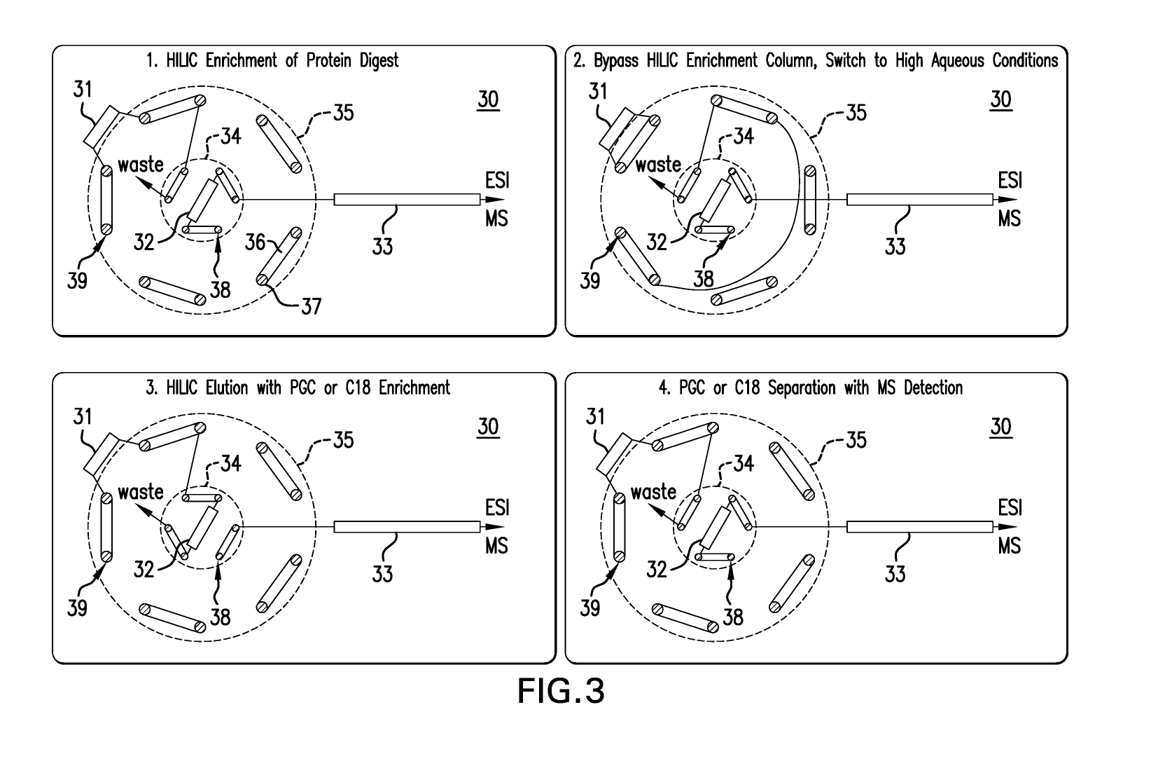

Embodiments of the invention relate to microfluidic HPLC chips that can facilitate the purification and analysis (e.g., HPLC/MS) of glycopeptides. FIG. 2 shows a functional-block diagram illustrating a microchip in accordance with one embodiment of the invention. As shown in FIG. 2, a microchip 20 may comprise three functional units: enrichment unit 21, concentration (trapping) unit 22, and separation/analysis unit 23. These different functional units may be connected by different flow paths at different stages of the operations (see discussion with reference to FIG. 3 below). Note that in some embodiments of the invention, the enrichment unit 21 and the trapping unit 22 may be the same (single) unit, as explained below.

In accordance with embodiments of the invention, the connections between different functional units 21, 22, and 23 may be controlled using microfluidic switches (or rotors). An example of a microfluidic chip comprising micro rotors and switches is found in Agilent mAb-glyco chip, which is designed for the analysis of carbohydrates associated with monoclonal antibody molecules. U.S. Patent Application Publication No. 2010/0190146 by Bynum et al. also discloses similar microfluidic devices for the analysis of glycans released from glycoproteins.

In accordance with embodiments of the invention, enrichment unit 21 may comprise a column having a stationary phase for binding carbohydrates that are attached to glycopeptides. A column for binding carbohydrates may be a hydrophilic column. Examples of hydrophilic columns include hydrophilic interaction chromatography (HILIC). The stationary phase of HILIC typically comprises polar materials, such as silica, cyano, amino, diol, etc. HILIC typically involves a mobile phase with high organic solvent content. A sample containing glycopeptides flowing into the enrichment unit 21 will have the glycopeptides enriched due to binding of their carbohydrate parts, while non-carbohydrate containing components (e.g., peptides or salts) will pass through.

In accordance with some embodiments of the invention, the microchip 20 may optionally include a trapping/concentration unit 22, which is used to concentrate glycopeptides to facilitate the subsequent analysis of the glycopeptides using HPLC. The main function of this concentration column is to trap and concentrate the glycopeptides. Therefore, a trapping column may comprise a stationary phase that can bind the carbohydrate parts and/or the peptide parts of the glycopeptides under the selected conditions.

In accordance with some embodiments of the invention, the trapping columns may be selected to bind the carbohydrate parts on the glycopeptides. Such columns for binding carbohydrates may include hydrophilic interaction liquid chromatography (HILIC) columns. In accordance with other embodiments of the invention, the trapping columns may be selected to bind the peptide parts of the glycopeptides. Such columns for binding peptides, for example, may include porous graphitic carbon (PGC) columns or reversed phase (e.g., C-4, C-8, or C-18) columns.

In accordance with some embodiments of the invention, both the enrichment and trapping columns may be selected for their abilities to bind the carbohydrate parts of the glycopeptides. In this case, the use of separate enrichment and trapping columns may not be necessary. Instead, these two functions (enrichment and trapping) may be performed using a single column, such as a HILIC column.

If separate enrichment and trapping columns are used, they may be selected for optimal binding of the glycopeptides under the selected conditions. For example, for enrichment using a HILIC column, a high organic content solvent is used. In this case, one may choose an orthogonal condition (e.g., an aqueous-based mobile phase) to perform the trapping using a reversed phase or porous graphite carbon (PGC) column. By using such orthogonal conditions, more impurities may be removed prior to the analysis.

In accordance with embodiments of the invention, the separation/analysis unit 23 is used to separate different glycopeptide components. The separation/analysis unit may use high performance liquid chromatography (HPLC) or electrophoresis, preferably HPLC. The HPLC columns used for glycopeptides separation and analysis, for example, may comprise reversed phase (e.g., C-1, C-4, C-8, or C-18) or PGC stationary phase. The individual components separated on this unit may be introduced into a mass spectrometer for identification. Any suitable mass spectrometer (e.g., time-of-flight (TOF) MS or electrospray ionization (ESI) MS) may be used with a micro HPLC chip of the invention.

FIG. 3 shows an embodiment of a microchip 30 in accordance with one embodiment of the invention. As shown, the micro chip 30 includes a HILIC column 31 for the enrichment of glycopeptides, a concentration (trapping) column 32 for trapping the glycopeptides, and an analytical HPLC column 33 for the separation of various glycopeptide components. The outlet of the HPLC column 33 is shown to interface with an ESI MS.

The microchip 30 also contains a plurality of inlet/outlet ports 36 that, in combination with channels 36 in a rotor or switch 34 or 35, can form different flow paths. An inlet/outlet port (or “port”) can be a hole, orifice, opening, or a combination of the above connected to a conduit (especially a short conduit), or the like, as long as the port allows fluid to pass from one end of the port to the other. The ports can be used to connect different parts of the device at different stages when microchip 30 is aligned with and coupled to appropriate channels on a rotor or switch. For example, microchip 30 can be fit on top of an inner rotor 34 and an outer rotor 25. The inner and/or outer rotors can be rotated so that different ports in microchip 30 are connected by channels in the rotor. The different channels in the rotor connect with different ports to form different flow paths. Thus, in combination with the channels on a rotor/switch, the inlet/outlet ports on the microchip 30 can provide different flow paths to connect or disconnect an enrichment column, a trapping column, and/or an analytical column. The uses of such rotors/switches in microfluidic devices are known in the art and will not be described in details here, see for example the operations of Agilent mAb-Glyco chip and U.S. Patent Application Publication No. 2010/0190146.

Although a rotor is described above as a switching element to change the fluid communication state of the columns in the microfluidic device, other switching elements (switches) may be used without departing from the scope of the invention. For example, a set of channels and valves can be used with the microfluidic device such that different columns may be put in the flow paths at different states.

In this particular example, the inner rotor 34 has channels for connecting to the inner 6 ports on the microchip 30, while the outer rotor 35 has channels for connecting with the 10 outer ports on the microchip 30. The particular numbers of ports may be altered to fit the particular need. Thus, these particular numbers are for illustration only and are not intended to limit the scope of the invention. Various connection configurations may be achieved by switching the inner rotor 34 and/or the outer rotor 35 to align with different inlet/outlet ports on the microchip 30, as described below.

FIG. 3 also illustrates the operation of this chip in accordance with embodiments of the invention. In the first step (FIG. 3, upper left panel), the HILIC enrichment column is switched in line. A glycopeptide sample is dissolved in high organic solvent and pumped by a sample pump 39 through the enrichment column 31. The glycopeptides in the sample bind to the HILIC enrichment column 31 due to the presence of carbohydrates, while other components (salts, non-glycosylated peptides, etc.) flow to the waste.

Then, in the second step (FIG. 3, upper right panel), the HILIC enrichment column 31 is switched off line, for example by switching the outer rotor 35. The flow path is then washed with a highly aqueous solvent.

Once the flow path is filled with an aqueous solvent, in the third step (FIG. 3, lower left panel), the outer rotor 35 is switched again to place the HILIC enrichment column 31 back in line with the LC flow. At the same time, the inner rotor 34 is switched to place the trapping column 32 (which may be a reversed phase column or a PGC column) downstream of the HILIC enrichment column 31. The high aqueous content of the mobile phase causes elution of the glycopeptides from the HILIC enrichment column 31. The eluted glycopeptides are subsequently trapped by the trapping column 33.

Finally, in step 4 (FIG. 3, lower right panel), the inner rotor 34 is switched to place the trapping column 32 in line with the analytical column 33. The analytical column 33 may be a reversed phase or PGC column. The components of the glycopeptides are separated on the analytical column 33 with a gradient of increasing organic solvent generated by the micropump 38. The separated glycopeptides may be sent to an ESI MS for analysis or identification.

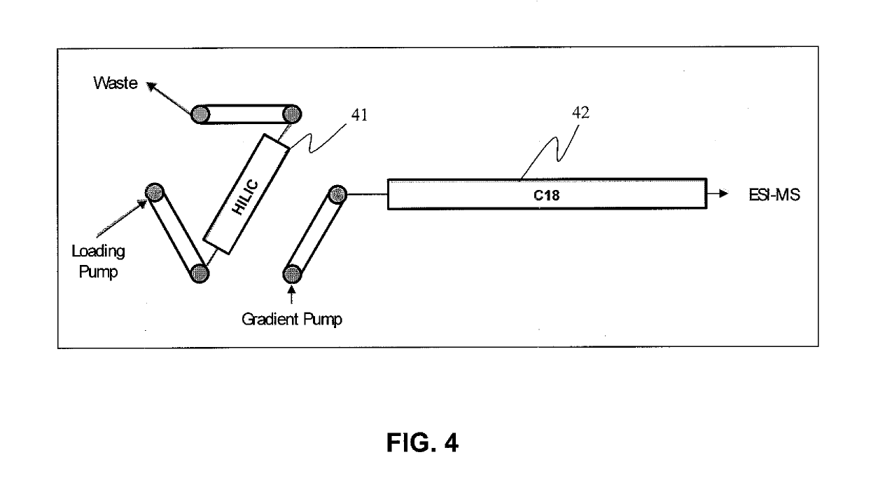

As noted above, in accordance with some embodiments of the invention, the enrichment and trapping of glycopeptides may use the same column, such as a HILIC column. FIG. 4 shows one such example, in which a HILIC column is used to enrich and trap the glycopeptides. Then, an analytical column (e.g., a reversed phase column C-18 column) is used to separate the glycopeptide components. Note that the specific columns shown here are for illustration only. One skilled in the art would appreciate that modifications and variations are possible without departing from the scope of the invention. For example, one may use a PGC column (instead of a reversed phase column) to separate/analyze the glycopeptides.

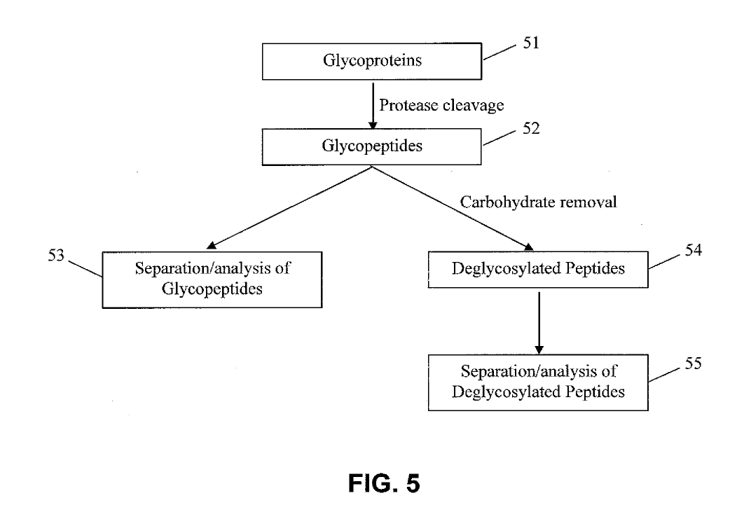

The above description discloses devices and methods for analyzing glycopeptides by taking advantage of the carbohydrate parts as handles for enriching and trapping the glycopeptides. While the presence of carbohydrates on the glycopeptides can have such advantages, these carbohydrates may present difficulties in the analysis. For example, the heterogeneity of carbohydrates may lower the MS peak signals by distributing the signal intensity over several peaks due to carbohydrate heterogeneity. In addition, the presence of carbohydrates on peptides is also known to substantially suppress the ionization of the glycopeptides, leading to low signal intensities in the MS spectra. Therefore, it is sometime desirable to be able to remove the carbohydrates from the glycopeptides before analysis. FIG. 5 outlines a general schematic that illustrates different methods for the analysis of glycoproteins or glycopeptides.

As shown in FIG. 5, glycoproteins 51 are typically hydrolyzed with proteases to produce smaller glycopeptide fragments, which are more amenable to separation and analysis. The protease cleavage may be accomplished with any suitable proteases. Commonly used proteases have known substrate specificities, such as trypsin, Glu-C, pepsin, etc. Among these proteases, trypsin and Glu-C are more commonly used. Trypsin is specific for the cleavage at the basic amino acid sites (i.e., lysine or arginine), while Glu-C cleaves after the glutamic acids.

The glycopeptide fragments may be separated and analyzed using the microchip devices described above (see e.g., FIG. 3). Alternatively, the glycopeptides may be further processed to remove the carbohydrate parts from the glycopeptides to produce deglycosylated peptides 54. Removal of carbohydrates can be accomplished with any known glycosidases, such as PNGase F, β-N-Acetyl-glucosaminidase, α-Fucosidase, β-Galactosidase, α-Galactosidase, α-Neuraminidase, α-Mannosidase, β-Glucosidase, β-Xylosidase, β-Mannosidase, Endo F₁, Endo F₂, Endo F₃, and Endo H.

Once the carbohydrate parts are removed from the glycopeptides, the remaining carbohydrate-free peptide fragments (i.e., deglycosylated peptides 54) can be separated and analyzed as normal peptides, using techniques known for regular peptides 55, such as LC/MS.

Some embodiments of the invention relate to microfluidic chips for the analysis of glycopeptides as deglycosylated peptides. The deglycosylated peptides may be generated with deglycosylation reactors, which may be included on the microfluidic chips. These microfluidic chips are similar to the Agilent mAb-Glyco chip. In accordance with embodiments of the invention, key features of these microfluidic HPLC chips include an integrated deglycosylation enzyme reactor, a deglycosylated peptide trapping column, and an analytical column.

The trapping column may be any suitable column that can bind peptides, such as reversed phase columns or PGC columns. In accordance with embodiments of the invention, the reversed phase trapping columns preferably are polymer-based reversed-phase columns because the mobile phases coming from the enzyme reactors may have neutral or slightly alkaline pH values.

The analytical columns for the analysis of the deglycosylated peptides may be PGC columns or reversed phase columns. For the reversed phase columns, they may be polymer based or silica based reversed phase columns.

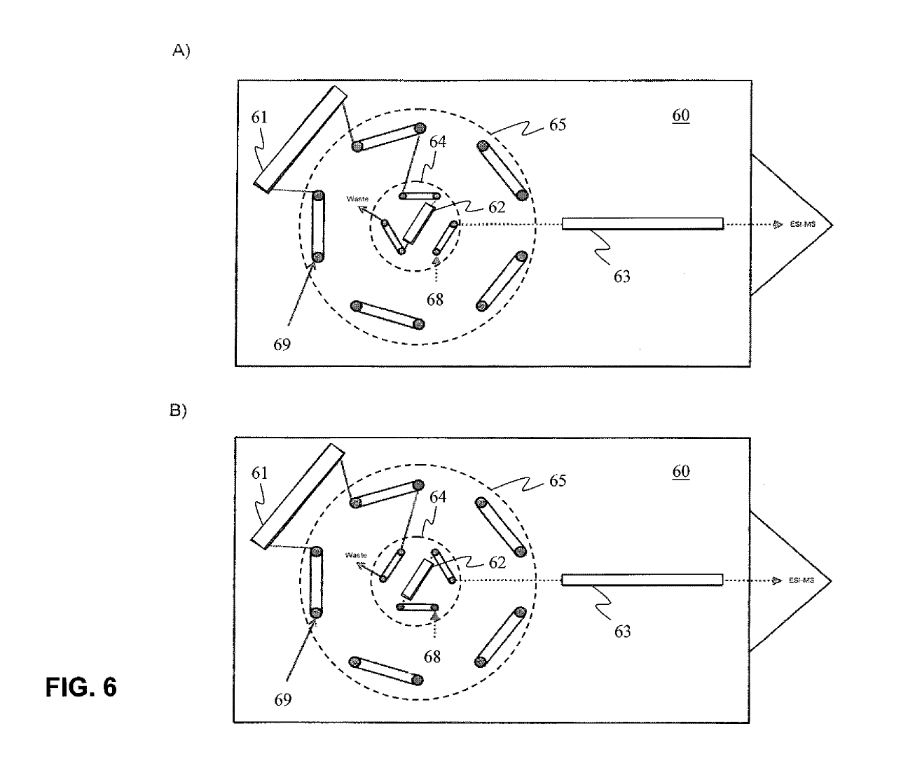

FIG. 6 panel (A) and FIG. 6 panel (B) show a microfluidic chip that includes a deglycosylation enzyme reactor in accordance with one embodiment of the invention. As shown, the microfluidic chip 60 comprises a deglycosylation column 61, a trapping column 62, and a separation/analysis column 63. Like other HPLC-chips, the devices may comprise inlet/outlet ports, which in combination with rotors may provide different flow paths. For example, an inner rotor (6-port) and an outer rotor (10-port) as described above may be used to connect with different inlet/outlet ports on the chips to control and switch different flow paths.

In accordance with embodiments of the invention, the deglycosylation column 61 comprises a solid support to which an enzyme is attached, wherein the enzyme is capable of cleaving carbohydrates from a glycoprotein. In most glycoproteins, the carbohydrate moiety is attached to the nitrogen of the amide group in asparagine residues (N-linked glycans), or the oxygen of the hydroxyl group in serine or threonine residues (O-linked glycans). Any enzyme that can cleave the carbohydrate moieties from glycoproteins can be used in the present invention, including enzymes that are specific for N-linked or O-linked glycans. These enzymes are known in the art and include, but are not limited to, PNGase F, β-N-Acetyl-glucosaminidase, α-Fucosidase, β-Galactosidase, α-Galactosidase, α-Neuraminidase, α-Mannosidase, β-Glucosidase, β-Xylosidase, β-Mannosidase, Endo F₁, Endo F₂, Endo F₃, and Endo H.

Materials and methods for immobilizing proteins to solid supports are also known in the art (see, e.g., Palm and Novotny, 2005). The solid support in the deglycosylation column 81 may be glass or polymer beads, or a monolithic medium (such as polymethacrylate, polystyrene, polyacrylamide, or the like). Examples of making immobilized glycosidase columns may be found in U.S. Patent Application Publication No. 2010/0190146, by Bynum et al., which discloses similar deglycosylation columns.

As noted above, the trapping column 62 may comprise any column that can trap deglycosylated peptides. Such columns may include hydrophilic interaction columns (HILIC columns), reversed phase columns (e.g., C-1, C-4, C-8, or C-18), or PGC columns.

In accordance with embodiments of the invention, the analysis column 63 may comprise any suitable column for the analysis of peptides known in the art. Examples of such analytical columns may include PGC columns, reversed phase columns (e.g., C-1, C-4, C-8, or C-18), or electrophoresis columns.

The operations of such microchips are described with reference to FIG. 6 panel (A) and FIG. 6 panel (B). Referring to FIG. 6 panel (A), a protein digest sample is loaded onto the microfluidic device 60 by a sample pump 69. Glycopeptides in the sample are deglycosylated in the enzyme reactor 81 (e.g., PNGase F reactor). This can be done in either a flow-through fashion or a “heart-cut” fashion. Deglycosylation on such immobilized enzyme reactors occurs efficiently. The reaction is typically complete within a few seconds to a few minutes. This is remarkable as compared to the traditional in-solution deglycosylation, which typically requires 12 hours or longer. The enzyme reactor (or column) 61 in this example is shown to contain PNGase F. However, any other suitable glycosidase may be used without departing from the scope of the invention.

The deglycosylated sample leaves the enzyme reactor 61 and flows over the trapping column 62. In accordance with embodiments of the invention, trapping column 62 is preferably a PGC or polymer-based reversed phase column, rather than a silica-based reversed phase column, because the mobile phase coming from the enzyme reactor 61 has a neutral or slightly alkaline pH for the deglycosylation reaction.

Once the enzymatic reaction is complete, the inner rotor 64 may be switched to place the trapping column 62 in line with the analytical column 63, as shown in FIG. 6 panel (B). In accordance with embodiments of the invention, the analytical column 63 is preferably a reversed phase column. Using a reversed phase analytical column 63, a gradient of increasing organic solvent may be generated by the analytical pump 68 to drive the separation of the peptides (now a mixture of deglycosylated glycopeptides and unmodified peptides). The separated peptides may be analyzed with downstream MS detection.

With a device of the invention as shown in FIG. 6, one can easily switch the deglycosylation reaction column in and out of line. Therefore, one can conveniently perform analysis using this device under different conditions with or without deglycosylation. Accordingly, confirmation of the presence of carbohydrates on peptide fragments can be easily accomplished, for example by MS peak shifts due to mass (m/z) changes.

Embodiments of the invention and their applications will now be further illustrated with the following examples. Note that these examples are for illustration only. One skilled in the art would appreciate that modifications and variations of these examples are possible without departing from the scope of the invention.

### Example 1

### Glycosite Analysis of RNAse B

In this example, RNAse B, a glycoprotein, is digested with trypsin. RNAse B protein contains a single glycosylation site (-NLT-) that may have different carbohydrates attached thereto. The carbohydrates are of the high mannose type, GlcNAc₂Manₓ, wherein x may be an integer from 5 to 9.

The tryptic digest is analyzed as glycopeptides (i.e., without deglycosylation) using a microship device, as shown in FIG. 3. The sample is first enriched on a HILIC column (see the enrichment column 31 in FIG. 3), then concentrated on a PGC trapping column (see the trapping column 32 in FIG. 3). Finally, the glycopeptides are separated on a PGC analytical column (see the analytical column 33 in FIG. 3).

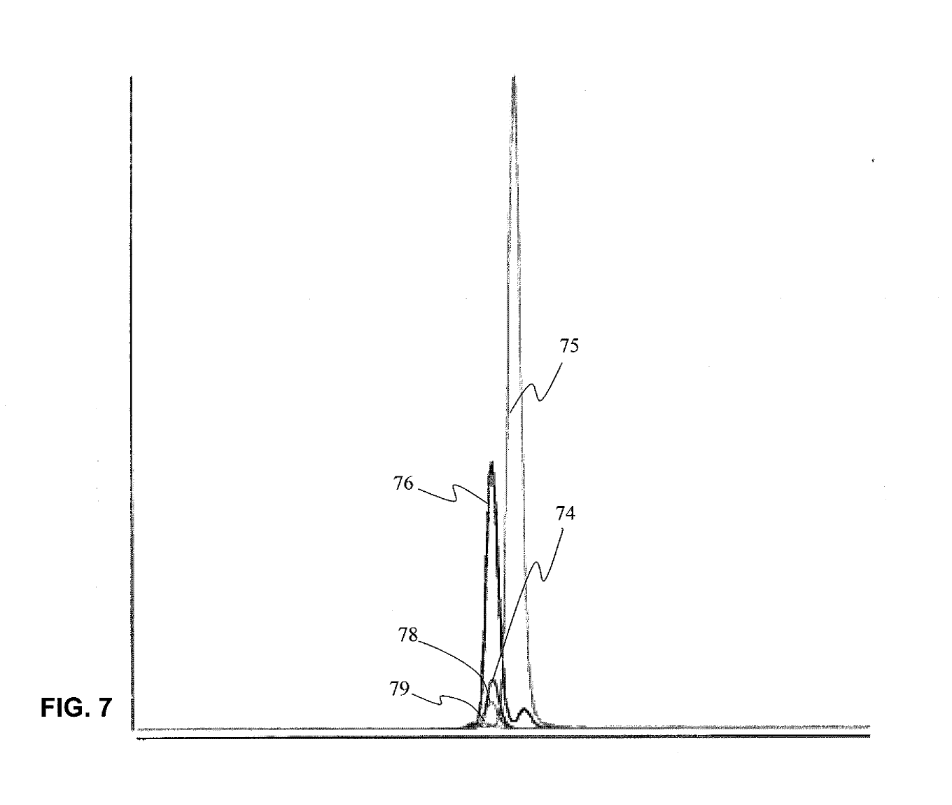

FIG. 7 shows the results of the analysis. As shown in FIG. 7, five different glycopeptide species are identified, representing five different types of high mannose glycans attached to the single glycosylation site on a single tryptic fragment. The five peaks in FIG. 7 are, respectively, 75 (GlcNAc₂Man₅), 76 (GlcNAc₂Man₆), 77 (GlcNAc₂Man₇), 78 (GlcNAc₂Man8₅), and 79 (GlcNAc₂Man₉). The successful separation of these five known species attests to the utility of embodiments of the invention. Similar results are obtained with Glu-C (instead of trypsin) digestion (data not shown).

### Example 2

### Glycosite Analysis of Haptoglobin

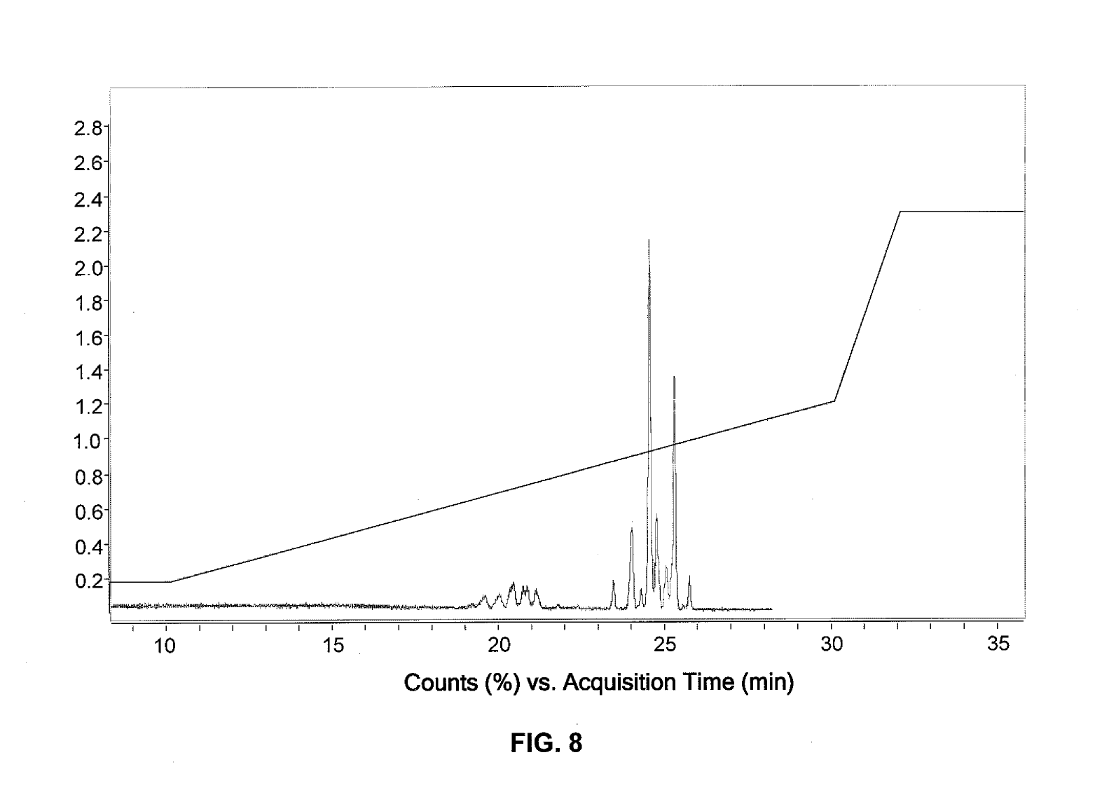

FIG. 8 shows results using a device of FIG. 4 for the characterization of a haptoglobin trypsin digest. Briefly, haptoglobin was digested by trypsin first. A 50 μg aliquot of haptoglobin was buffer exchanged using a molecular weight cut off membrane into 100 mM ammonium bicarbonate pH 8.0, denatured with trifluoroethanol (TFE), and reduced and alkylated with DTT and iodoacetamide. TFE and other salts were removed and the protein was digested with trypsin overnight at 37° C.

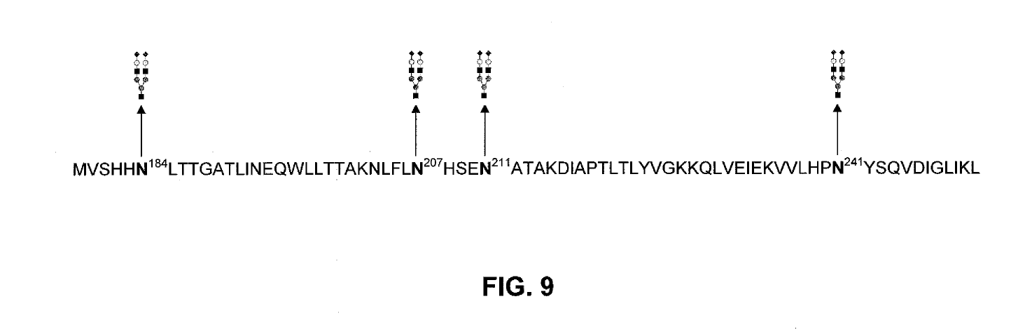

The tryptic digest was enriched on a HILIC column and then separated on a reversed phase C-18 column using a micro HPLC chip as shown in FIG. 4. As shown in the results of FIG. 8, various glycopeptides are well resolved and can be identified by MS. FIG. 9 shows the partial peptide sequence of haptoglobin and its four N-glycosylation sites.

Similar experiments have also been performed using Glu-C, instead of trypsin (data not shown). In addition, similar experiments have also been conducted using erythropoietin and cleavage with trypsin or Glu-C (data not shown). Similar results are obtained and all different species of the glycopeptides can be identified.

### Example 3

### Glycosite Analysis of Fetuin

The above examples show the utility of microchips of the invention for the analysis of glycopeptides that still contain carbohydrates (i.e., without deglycosylation). This example and the following examples demonstrate the utility of microchips of the invention that incorporate deglycosylation reactions for the analysis of glycopeptides, using a device as shown in FIG. 6.

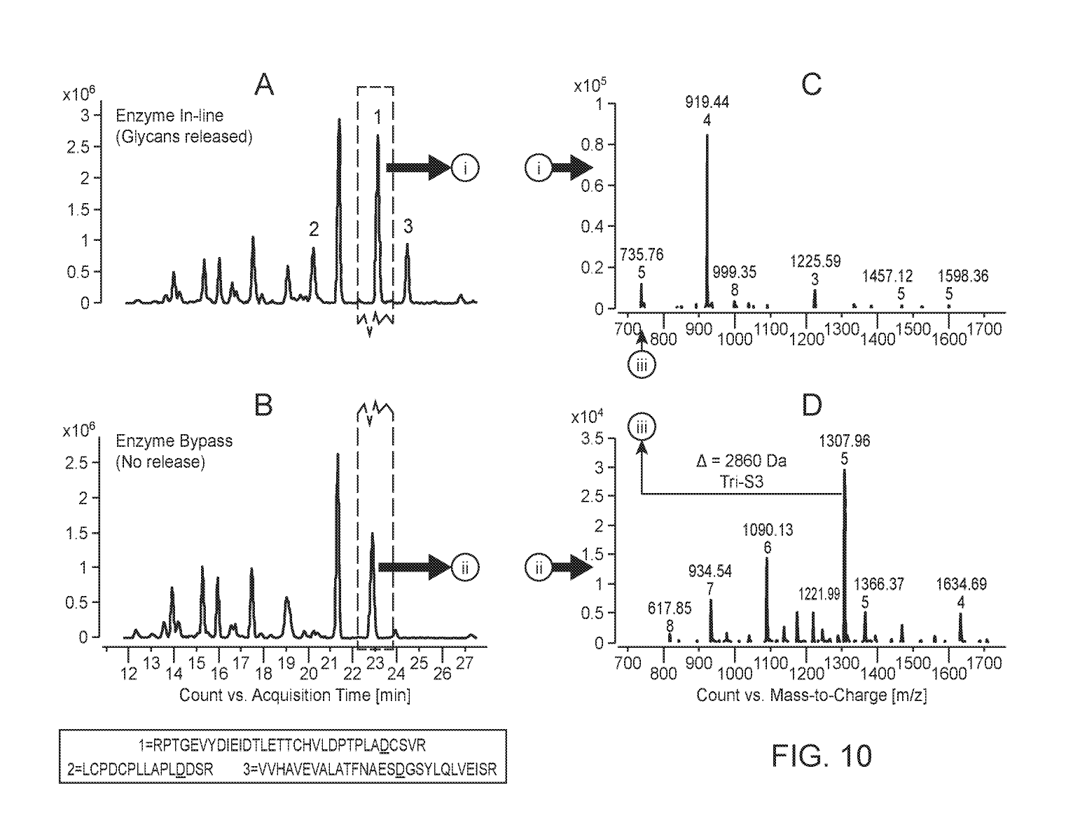

FIG. 10 shows results of on-line deglycosylation and analysis using a device of FIG. 6 to analyze fetuin. Fetuin is a glycoprotein containing three N-glycosylation sites that can be occupied by a number of complex N-glycans. Successful operation of the device was demonstrated by the detection of all three deglycosylated peptides expected from the sample, as shown in FIG. 10. The peptide sequences are shown below the figure. Note that glycosylated Asn residues would be converted to Asp residues (N→D) after deglycosylation.

FIG. 10 panel (A) (top left graph) shows a total compound chromatogram of fetuin tryptic digest deglycosylated using a “heart-cut” approach. FIG. 10 panel (B) (bottom left graph) shows a total compound chromatogram of fetuin tryptic digest with no deglycosylation (i.e., the carbohydrates are still attached to the peptides). Note that three new peaks appear in the deglycosylated (FIG. 10 panel (A)) chromatogram, corresponding to glycopeptides that have been deglycosylated (N-glycosylation sites 1, 2, and 3).

The outlined area (dotted box) highlights peaks corresponding to glycosylation at site 1, which show a deglycosylated peptide (FIG. 10 panel (A)) which appears at a slightly later retention time than its glycosylated form (FIG. 10 panel (B)).

The mass spectra at right correspond to the peptide containing site 1 in its deglycosylated form (FIG. 10 panel (C)) and glycosylated form (FIG. 10 panel (D)), respectively. The deglycosylated MS spectrum is much simpler, as a result of glycan removal. The mass difference between the 5+charge states observed in each spectrum corresponds to a triantennary, trisialylated glycan (Tri-S3), as expected.

### Example 4

### Quantitative Deglycosylation of Human Transferrin

The deglycosylation reactors of the invention are unexpectedly efficient. While deglycosylation typically requires many hours (typically, 12 hours or more) in a conventional solution reaction. The immobilized enzymes of the invention can remove carbohydrates within minutes or less. More importantly, such immobilized reactors are very efficient and can achieve complete reaction in most cases.

The ability of a microchip of the invention to perform quantitative (100%) or substantially quantitative deglycosylation of a given site on a glycopeptide is demonstrated in this example. Human transferrin, a glycoprotein with two N-glycosites, is digested and analyzed with or without deglycosylation.

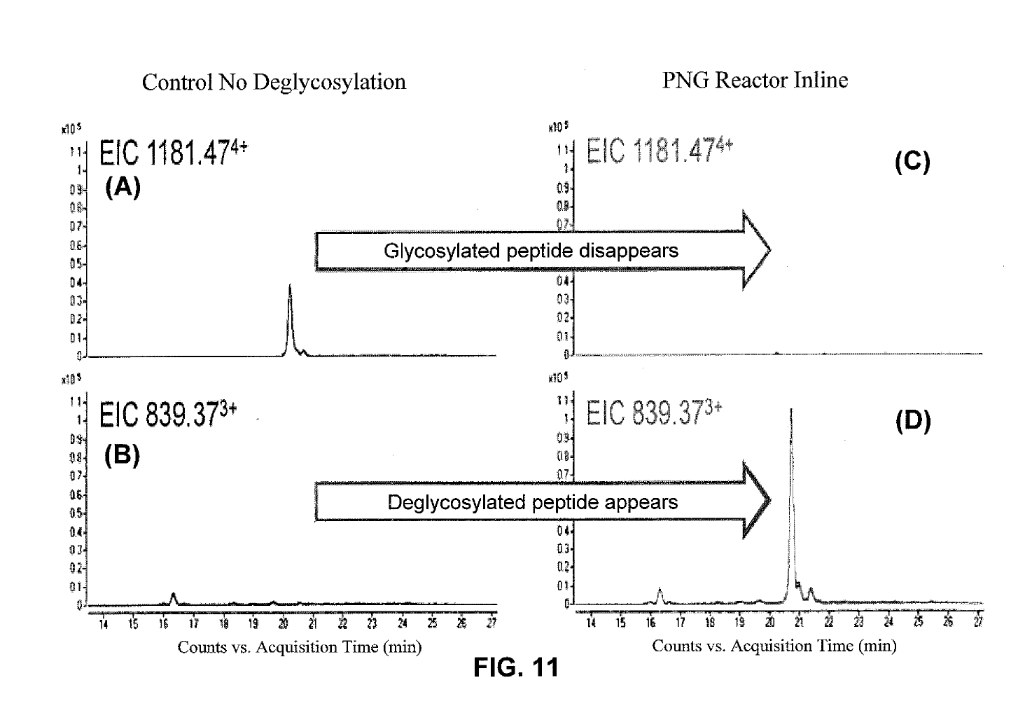

FIG. 11 shows an on-line deglycosylation of transferrin digest. Extracted ion chromatograms matching the m/z of an abundant glycopeptide and the m/z of the deglycosylated version of that same glycopeptide were produced from LC/MS runs of transferrin with and without deglycosylation.

As shown, the peak for the glycopeptide with m/z 1181.47 (4+) (FIG. 11 panel (A)) disappears upon deglycosylation (FIG. 11 panel (C)), indicating quantitative release of the carbohydrate. Concomitant with this is the appearance of m/z 839.37 (3+) (FIG. 11 panel (B)→FIG. 11 panel (D)). The m/z 839.37 (3+) corresponds to the mass of the glycopeptide (m/z 1181.47 (4+)) minus a biantennary, disialylated N-glycan. These results show that the deglycosylation is complete.

### Example 5

### Confirmation of Deglycosylation Reaction

The above examples demonstrate the utility of deglycosylation in the characterization of glycoproteins or glycopeptides. The removal of carbohydrates from the glycopeptides alters their properties such that one can use different columns for the trapping and analysis of the deglycosylated peptides. More importantly, deglycosylation increases the sensitivity of MS analysis and simplifies the peaks. These changed properties can be used to confirm that the deglycosylation has occurred, see for example the results shown in FIG. 11.

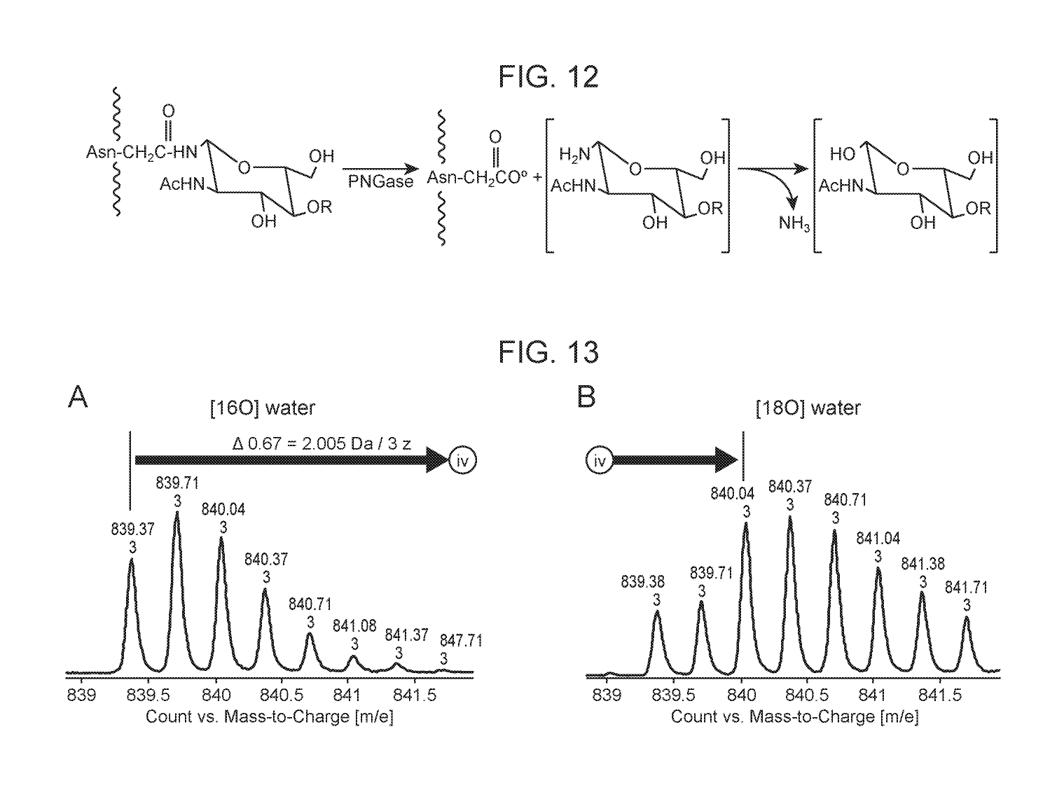

In addition, deglycosylation may also be confirmed based on its reaction mechanism. For example, FIG. 12 outlines the reaction mechanism of a glycosidase reaction. As shown, the N-glycan is hydrolyzed in such a manner that the N atom from the asparagine residue ends up with the carbohydrate part. The initial product is carbohydrate glycosylamine. The glycosylamine is not stable and will be hydrolyzed to produce the corresponding —OH species (i.e., free reducing end).

Thus, after a glycosidase reaction, the asparagine residue is converted into an aspartic acid residue, which is accompanied by a mass shift of 0.984 Da and is detectable by MS. Unfortunately, the 0.984 Da mass shift is the same as deamidation of any amide group on the peptide, which can confound the identification of glycosylation site by MS approaches. As a workaround, one may perform the deglycosylation in H₂¹⁸O water, which will impart an ¹⁸O isotope into the aspartic residue with an increase of 2.989 Da, which is easily distinguished from a deamidation reaction.

FIG. 13B shows results of deglycosylation in H₂¹⁸O using the described device, as shown in FIG. 6. For this experiment, the tryptic digest of transferrin was diluted in H₂¹⁸O before injection onto the HPLC-chip device.

FIG. 13 panel (A) shows results of a glycosylated peptide from transferrin hydrolyzed by PNGase in standard H₂O. FIG. 13 panel (B) shows results of the same glycosylated peptide from transferrin hydrolyzed by PNGase in H₂¹⁸O. Note that the isotopic distribution of the deglycosylated peptides is altered by the incorporation of one ¹⁸O atom as a result of the experimental condition. This isotope shift can be readily detected with MS. This example demonstrates that one can conveniently confirm the deglycosylation reaction using a microfluidic chip of the invention.

The above examples demonstrate that microfluidic chips of the invention are useful for the analysis of glycoproteins and glycopeptides, with or without deglycosylation. When glycopeptides are analyzed without deglycosylation, embodiments of the invention take advantages of the carbohydrate parts on the glycopeptides to enrich and concentrate the glycopeptide for analysis on microfluidic HPLC columns. With some embodiments of the invention, very efficient deglycosylation reactors are used to deglycosylate the glycopeptides so that they can be analyzed without interference from the carbohydrate parts. Because the devices of the invention can be easily changed from one with a deglycosylation reactor to one without a deglycosylation reactor, one can easily perform analysis to compare reactions with and without deglycosylation to confirm the glycosites.

Advantages of embodiments of the invention may include one or more of the following. Embodiments of the invention provide microfluidic chips that can facilitate the analysis of glycopeptides. The use of these devices is convenient and would not waste sample. In addition, because the enzymatic reactions are markedly more efficient using the immobilized enzyme reactors and the operation is simple, one would save time and costs using these devices.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A microfluidic device for glycopeptide analysis, comprising:
an enrichment column comprising a stationary phase capable of binding carbohydrates;
a trapping column comprising a stationary phase capable of binding peptides, wherein the trapping column is configured to be connected downstream of the enrichment column;
a separation column comprising a stationary phase capable of separating peptides, wherein the separation column is configured to be connected downstream of the trapping column; and
a plurality of ports configured to work with a switching device to form a plurality of flow paths, wherein one of the plurality of flow paths allows the trapping column to be in fluid communication with the separation column.

2. The microfluidic device of claim 1, wherein the enrichment column comprises a hydrophilic interaction (HILIC) stationary phase.

3. The microfluidic device of claim 1, wherein the trapping column comprises a hydrophilic interaction (HILIC) stationary phase, a reversed phase stationary phase, or a porous graphitic carbon (PGC) stationary phase.

4. The microfluidic device of claim 1, wherein the separation column comprises a reversed-phase stationary phase or a porous graphitic carbon (PGC) stationary phase.

5. The microfluidic device of claim 4, wherein the reversed-phase stationary phase comprises a C-18 silica-based stationary phase.

6. A microfluidic device for glycopeptide analysis, comprising:
a deglycosylation column comprising a solid support having a glycosidase immobilized thereto;
a trapping column comprising a stationary phase capable of binding peptides, wherein the trapping column is configured to be connected downstream of the deglycosylation column;
a separation column comprising a stationary phase capable of separating peptides, wherein the separation column is configured to be connected downstream of the trapping column; and
a plurality of ports configured to work with a switching device to form a plurality of flow paths, wherein one of the plurality of flow paths allows the trapping column to be in fluid communication with the separation column.

7. The microfluidic device of claim 6, wherein the glycosidase is one selected from PNGase F, β-N-Acetyl-glucosaminidase, α-Fucosidase, β-Galactosidase, α-Galactosidase, α-Neuraminidase, α-Mannosidase, β-Glucosidase, β-Xylosidase, β-Mannosidase, Endo F₁, Endo F₂, Endo F₃, or Endo H.

8. The microfluidic device of claim 6, wherein the glycosidase is PNGase F.

9. The microfluidic device of claim 8, wherein the trapping column is a polymer-based reversed phase column.

10. The microfluidic device of claim 9, wherein the separation column is a silica-based reversed phase column.

11. A method for glycopeptide analysis using a microfluidic device comprising an enrichment column capable of binding carbohydrates, a trapping column capable of binding peptides, and a separation column, the method comprising:
applying a sample of glycopeptides to the microfluidic device;
enriching the glycopeptides on the enrichment column;
trapping the glycopeptides from the enrichment column on the trapping column;
eluting the glycopeptides from the trapping column into the separation column; and
separating the glycopeptides on the separation column.

12. The method of claim 11, wherein the enrichment column comprises a hydrophilic interaction (HILIC) stationary phase.

13-15. (canceled)

16. The method of claim 11, wherein the trapping column is a polymer-based reversed phase column.

17. The method of claim 16, wherein the separation column is a silica-based reversed phase column.
